(19) United States
(12) Patent Application Publication
(10) Pub. No.: US 2014/0349960 A1
(43) Pub. Date: Nov. 27, 2014

(54) COMPOSITION FOR BODY FAT CONSUMPTION

(71) Applicant: TAIWAN HOPAX CHEMS. MFG. CO, LTD., Kaohsiung City (TW)

(72) Inventors: Han-Fen HUANG, Kaohsiung City (TW); Li-Jane HER, Kaohsiung City (TW)

(73) Assignee: TAIWAN HOPAX CHEMS. MFG. CO, LTD., Kaohsiung City (TW)

(21) Appl. No.: 14/286,084

(22) Filed: May 23, 2014

Related U.S. Application Data
(60) Provisional application No. 61/826,606, filed on May 23, 2013.

(51) Int. Cl. A61K 31/737

(52) U.S. Cl. CPC A61K 31/737
USPC 514/55

(57) ABSTRACT

The present invention related to a composition comprising an aqueous soluble-chitosan and a pharmaceutically acceptable carrier. Said composition can be used to increase lipase activity while having no harm in animal physiology. Together with the well known biocompatibility of chitosan, the present invention proves that the aqueous soluble-chitosan may be a potential candidate for body weight control.

### BACKGROUND

1. Technical Field

The present invention is related to a composition for body fat consumption, especially by using chitosan.

2. Description of Related Art

Obesity is the most common lifestyle-related diseases in modern society. More and more evidences reveal that obesity is a key risk factor in lots of diseases such as heart disease, diabetes, high blood pressure, cancer, etc. In consideration of improving citizen health and relieving the financial burden of national health insurance, the government keeps advocating the importance of controlling body weight. In this regards, some health indexes, such as Body Mass Index (BMI) and Waist-Hip Ratio (WHR) are used as a measurement for body weight control.

It has been gathered long-time interests to develop novel drugs or reagents to help people lose weight. Unfortunately, there is still lack of such drugs or reagents with high efficiency and low side effects so far. The situation could be worse as since the importance of body weight control has been acknowledged while no effective and safe drugs are available, people may easily believe in unconfirmed folk prescription and taking some unproved drugs before permission from the authority. It not only has no help in losing weight but also puts their health and life in dangerous. Therefore, there is a constant demand for a composition that has high efficiency in body fat consumption and low side effects.

### SUMMARY

One object of the present invention is to provide a novel composition has good efficiency in body fat consumption and has fewer side effects to animal physiology.

In order to achieve the above objects, the present invention provides a composition for body fat consumption, comprising: 0.1 to 80 wt % of an aqueous soluble-chitosan; and 1 to 50 wt % of a pharmaceutically acceptable carrier.

The present invention also provides a method for increasing the activity of lipase, comprising: applying a subject in need an effective amount of an aqueous soluble-chitosan.

Preferably, said aqueous soluble-chitosan has a molecular weight of 0.3 to 1,500 kDa; more preferably, said aqueous soluble-chitosan has a molecular weight of 0.5 to 300 kDa.

Preferably, said aqueous soluble-chitosan is a chitosan modified by alkyl sultone. More preferably, said alkyl sultone is 1,3-propanesultone, 1,4-propylenesultone, 1,4-butanesultone, 2,4-butanesultone, or a mixture thereof

Preferably, said aqueous soluble-chitosan is a sulfonic acid-modified chitosan.

Preferably, said effective amount is 1 to 500 mg/kg BW.

Preferably, said lipase is adipose lipase.

To sum up, the present invention surprisely found that the aqueous soluble-chitosan has superior effect on body fat consumption which may be due to its ability to increase lipase activity. Furthermore, the aqueous soluble-chitosan shows no harm in animal physiology. Together with the well known biocompatibility of chitosan, the present invention proves that the aqueous soluble-chitosan may be a potential candidate for body weight control.

### BRIEF DESCRIPTION OF THE DRAWINGS

FIG. 1 shows the effects of AS-CH on the body weight gain percent in HFD rats.

FIG. 2 shows the effects of AS-CH on the lipase activity in HFD rats.

### DETAILED DESCRIPTION

### Embodiment 1

### Preparation of Aqueous Soluble-Chitosan

The aqueous soluble-chitosan of the present invention is chitosan that is modified by alkyl sultone. Examples of alkyl sultone include but not limited to 1,3-propanesultone, 1,4-propylenesultone, 1,4-butanesultone, 2,4-butanesultone, or a mixture thereof. More specifically, the aqueous soluble-chitosan of the present invention is a sulfonic acid-modified chitosan. For example, the aqueous soluble-chitosan is alkyl sulfonic acid-modified chitosan. The alkyl sulfonic acid-modified chitosan may be fabricated by the following procedures:

161 gram of chitosan (with molecular weight of 140,000) was put into a flask, and 700 ml of methanol was added in to obtain a mixture. The mixture was heated at 65 to 67° C., and 122 gram of 1,3-propanesultone was slowly dropped in while stirring. The mixture was kept refluxing for 4 hours after all 1,3-oxathiolane was added in. Then the flask was cooled down to room temperature, and product (alkyl sulfonic acid-modified chitosan) was collected by filtering. The product was washed by methanol from several times and dried overnight in a vacuum oven. The dried product was weighted 282 gram. The yield rate of the alkyl sulfonic acid-modified chitosan was 99.7%.

### Embodiment 2

### Experiment Design of Animal Model

The experiment was conducted by using 4-weeks old weaned Sprague-Dawley rats (purchased from BioLASCO Taiwan Co., Ltd). 64 rats were randomly separated into 8 groups. Each group had 8 rats. The experimental rats were maintained in plastic cages with free access to food and water. The temperature of those cages were kept at 25±1° C., and the day-night cycle was 12 hours per day. For experiments, rats were fed with normal diet (AIN-93G, ICN Biomedicals, Costa Mesa, Calif., USA) or high calorie diet to induce obesity (Modify AIN-93G high fat diet, 20% lipid) for 4 weeks before the administration of aqueous soluble-chitosan. Beginning from the fifth week, the experimental rats were fed with various dosages (10 or 25 mg/kg body weight) of unmodified chitosan and aqueous soluble-chitosan at every Monday, Wednesday, Friday and Saturday. Chitosan used was resolved in sterile water for feeding. One group of normal diet and one group of high calorie diet were instead fed with water as control. The experimental period was 8 to 12 weeks (the experiments were stopped depending on when the body weight of control group and test group show significant difference). The body weight and feeding amount (food intake) of the animals under experiments were measured and recorded every week.

The experimental animals were to be sacrificed by applying carbon dioxide after 12 weeks. Before sacrificing, those animals were starved for 12 hours. Collecting rats' blood, livers, hearts, spleens, kidneys, and colons for biochemical analysis and pathology study. Also, the adipose tissues of rat were collected for determining the amount of body fat and analyzing the activity of lipase.

### Embodiment 3

### Experimental Results

### [Blood Lipid Analysis]

The concentration of triglyceride (TG), total cholesterol (TC), high density lipoprotein (HDL) and low density lipoprotein (LDL) in blood were examined. Briefly, the blood to be examined was collected from abdominal aorta and was examined by enzymatic method and colorimetry method. The results are showed in the following Table 1 (ND: normal diet; HFD: high fat diet; CH: chitosan (unmodified); AS-CH: aqueous soluble-chitosan (the present invention); L: low dosage (10 mg/kg BW); H: high dosage (25 mg/kg BW)).

TABLE 1
Effects of AS-CH on the blood lipid in HFD rats

| | TG | TC | HDL | LDL |
|---|---|---|---|---|
| | mg/dL | | | |
| ND | 58 ± 8.4^{d} | 55 ± 2.5^{abc} | 56.9 ± 7.4^{a} | 10.1 ± 2.7^{ab} |
| HFD | 103 ± 3.0^{a} | 69 ± 11.0^{a} | 47.1 ± 10.0^{ab} | 15.0 ± 3.2^{a} |
| AS-CH or | | | | |
| CH | | | | |
| CH (L) | 88 ± 5.3^{abc} | 56 ± 8.5^{abc} | 43.9 ± 6.0^{b} | 15.4 ± 3.6^{a} |
| CH (H) | 96 ± 3.8^{ab} | 56 ± 5.6^{abc} | 45.9 ± 7.0^{ab} | 14.4 ± 3.5^{a} |
| AS-CH (L) | 81 ± 9.9^{bc} | 45 ± 5.7^{c} | 40.8 ± 7.7^{b} | 8.3 ± 1.5^{b} |
| AS-CH (H) | 74 ± 8.9^{cd} | 48 ± 6.0^{bc} | 46.2 ± 8.5^{ab} | 8.3 ± 1.2^{b} |

SD rat was orally administered with various dosages AS-CH (10 or 25 mg/kg BW) for 8 wks. Data is expressed as means ± SD (n = 8). Significance of difference in activities of different compounds was evaluated by Tukey's test statistical analysis. Different superscript letters^{a,b,c} blood lipid are statistically different from each other (p < 0.05).

The results showed that by applying the aqueous soluble-chitosan of the present invention, the blood TG, TC and LDL was lowered down while HDL (so called ‘good lipoprotein’) remained.

### [Liver TG & TC Analysis]

After the blood was collected, the liver was washed by saline and the TG and TC therein were extracted by the method taught by Folch et al. (Folch et al., 1957) for analysis. The results are showed in the following Table 2 (ND: normal diet; HFD: high fat diet; CH: chitosan (unmodified); AS-CH: aqueous soluble-chitosan (the present invention); L: low dosage (10 mg/kg BW); H: high dosage (25 mg/kg BW)).

TABLE 2
Effects of AS-CH on the liver TG and TC in HFD rats

| | |
|---|---|
| TG | TC |

mg/dL

| | | |
|---|---|---|
| ND | 109 ± 11^{b} | 16.0 ± 2.3^{c} |
| HFD | 153 ± 15^{a} | 26.4 ± 1.7^{ab} |
| AS-CH or CH | | |
| CH (L) | 152 ± 21^{a} | 22.0 ± 7.0^{bc} |
| CH (H) | 135 ± 16^{ab} | 25.8 ± 2.2^{abc} |
| AS-CH (L) | 119 ± 12^{b} | 19.6 ± 7.4^{bc} |
| AS-CH (H) | 114 ± 12^{b} | 22.0 ± 2.0^{bc} |

SD rat was orally administered with various dosages AS-CH (10 or 25 mg/kg BW) for 8 wks. Data is expressed as means ± SD (n = 8). Significance of difference in activities of different compounds was evaluated by Tukey's test statistical analysis. Different superscript letters^{a,b,c} blood lipid are statistically different from each other (p < 0.05).

The results indicated that the TG and TC level of the group administrated with the aqueous soluble-chitosan of the present invention were recovered back to normal standard as comparing with the control group of normal diet.

### [Blood Sugar Analysis]

After starvation for 12 hours, the experimental animals were anesthetized by ether. Then the blood was collected from abdominal aorta for analyzing the blood sugar level by enzymatic method and colorimetry method. The results are showed in the following Table 3 (ND: normal diet; HFD: high fat diet; CH: chitosan (unmodified); AS-CH: aqueous soluble-chitosan (the present invention); L: low dosage (10 mg/kg BW); H: high dosage (25 mg/kg BW)).

TABLE 3
Effects of AS-CH on the blood sugar in HFD rats

| | Blood sugar |
|---|---|
| | mg/dL |
| ND | 163 ± 27.7 |
| HFD | 185 ± 19.6 |
| AS-CH or CH | |
| CH (L) | 172 ± 15.4 |
| CH (H) | 173 ± 23.0 |
| AS-CH (L) | 191 ± 28.7 |
| AS-CH (H) | 168 ± 37.9 |

SD rat was orally administered with various dosages AS-CH (10 or 25 mg/kg BW) for 8 wks. Data is expressed as means ± SD (n = 8).

### [Hepatic & Kidney Function Analysis]

The AST, ALT, creatinine, uric acid were detected by enzymatic method and colorimetry method for determining the hepatic function. The results are showed in the following Table 4 (ND: normal diet; HFD: high fat diet; CH: chitosan (unmodified); AS-CH: aqueous soluble-chitosan (the present invention); L: low dosage (10 mg/kg BW); H: high dosage (25 mg/kg BW)).

TABLE 4
Effects of AS-CH on the function
of hepatic and kidney in HFD rats

| | AST | ALT | Creatinine | Uric acid |
|---|---|---|---|---|
| | U//L | | mg/dLc | |
| ND | 162 ± 27.7 | 47.5 ± 8.01 | 0.53 ± 0.07 | 3.44 ± 0.98 |
| HFD | 145 ± 35.3 | 49.0 ± 7.78 | 0.51 ± 0.08 | 4.56 ± 0.69 |
| AS-CH or | | | | |
| CH | | | | |
| CH (L) | 176 ± 41.6 | 49.6 ± 9.24 | 0.54 ± 0.05 | 4.90 ± 0.88 |
| CH (H) | 159 ± 32.2 | 44.1 ± 7.71 | 0.50 ± 0.08 | 4.27 ± 0.91 |
| AS-CH (L) | 172 ± 34.9 | 66.6 ± 18.99 | 0.54 ± 0.05 | 4.77 ± 0.38 |
| AS-CH (H) | 161 ± 38.1 | 58.1 ± 25.68 | 0.47 ± 0.05 | 4.45 ± 0.71 |

SD rat was orally administered with various dosages AS-CH (10 or 25 mg/kg BW) for 8 wks. Data is expressed as means ± SD (n = 8).

It was showed that the administration of the aqueous soluble-chitosan of the present invention had no harm on the liver and kidney function of the experimental animals.

### [Ketone Bodies and Electrolyte Balance Analysis]

After starvation for 12 hours, the experimental animals were anesthetized by ether. Then the blood was collected from abdominal aorta for analyzing the concentration of ketone bodies, Na⁺ ion and K⁺ ion in the blood by enzymatic method and colorimetry method. The results are showed in the following Table 5 (ND: normal diet; HFD: high fat diet; CH: chitosan (unmodified); AS-CH: aqueous soluble-chitosan (the present invention); L: low dosage (10 mg/kg BW); H: high dosage (25 mg/kg BW)).

TABLE 5
Effects of AS-CH on the electrolyte
balance and ketone bodies in HFD rats

| | Na⁺ | K⁺ | Ketone bodies |
|---|---|---|---|
| | mEq/L* | | nmole |
| ND | 150 ± 3.06^{ab} | 7.73 ± 1.4^{ab} | 0.96 ± 0.34 |
| HFD | 151 ± 1.33^{a} | 6.92 ± 0.5^{ab} | 1.04 ± 0.35 |
| AS-CH or CH | | | |
| CH (L) | 150 ± 0.92^{ab} | 7.23 ± 1.0^{ab} | 1.25 ± 0.43 |
| CH (H) | 149 ± 1.25^{ab} | 8.16 ± 0.7^{a} | 1.02 ± 0.28 |
| AS-CH (L) | 148 ± 1.63^{b} | 8.39 ± 0.3^{a} | 1.08 ± 0.51 |
| AS-CH (H) | 150 ± 1.33^{ab} | 7.30 ± 1.2^{ab} | 0.99 ± 0.13 |

*mEq/L: molar concentration of ion per liter
SD rat was orally administered with various dosages AS-CH (10 or 25 mg/kg BW) for 8 wks. Data is expressed as means ± SD (n = 8). Significance of difference in activities of different compounds was evaluated by Tukey's test statistical analysis. Different superscript letters^{a,b,c} electrolyte balance are statistically different from each other (p < 0.05).

By summarizing with the data in above Table 3, Table 4, and Table 5, the administration of the aqueous soluble-chitosan of the present invention had no effects on blood sugar, the ketone bodies and electrolyte balance in the blood. Also, it was showed that the administration of the aqueous soluble-chitosan of the present invention had no harm on the liver and kidney function of the experimental animals.

### [Analysis for Food Intake, Body Weight, and Feed Availability]

As mentioned in the aforementioned paragraphs, the body weight and food intake of the experimental animals were recorded regularly. Based on the recorded body weight, the change in body weight was calculated. Moreover, the feed efficiency was also calculated according to the formula: Feed Efficiency=(Weight Gain/Food Intake)×100%. Also, the organ weight was examined.

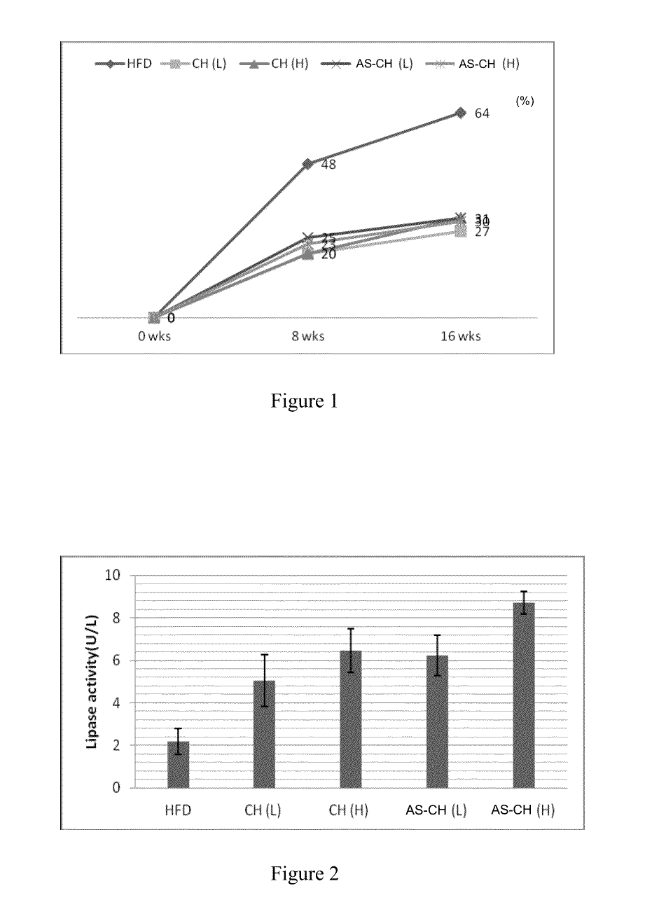

The results are showed in the following Table 6, Table 7 and FIG. 1, Table 8, and Table 9 (ND: normal diet; HFD: high fat diet; CH: chitosan (unmodified); AS-CH: aqueous soluble-chitosan (the present invention); L: low dosage (10 mg/kg BW); H: high dosage (25 mg/kg BW)).

TABLE 6
Effects of AS-CH on the food intake and body weight in HFD rats

| | Food intake * | | Body weight | | |
|---|---|---|---|---|---|
| | (g/day) | | (g) | | |
| | 8 wks | 16 wks | 0 wks | 8 wks | 16 wks |
| ND | 30.8 | 29.4 | 111 ± 8 | 352 ± 18^{c} | 459 ± 36^{c} |
| HFD | 21.5 | 22.6 | 116 ± 5.5 | 520 ± 31^{a} | 753 ± 24.^{a} |
| AS-CH or CH | | | | | |
| CH (L) | 19.9 | 16.9 | 107 ± 10.8 | 421 ± 31^{b} | 585 ± 57^{b} |
| CH (H) | 21.4 | 16.3 | 113 ± 6.1 | 424 ± 31^{b} | 602 ± 51^{b} |
| AS-CH (L) | 21.8 | 16.9 | 115 ± 7.3 | 440 ± 14^{b} | 602 ± 61^{b} |
| AS-CH (H) | 20.1 | 13.5 | 110 ± 15.0 | 432 ± 21^{b} | 598 ± 62^{b} |

* Data was averaged of groups
SD rat was orally administered with various dosages AS-CH (10 or 25 mg/kg BW) for 8 wks. Data is expressed as means ± SD (n = 8). Significance of difference in activities of different compounds was evaluated by Tukey's test statistical analysis. Different superscript letters^{a,b,c} body weight are statistically different from each other (p < 0.05)

TABLE 7
Effects of AS-CH on the body weight gain percent in HFD rats

| | Body weight gain (%) | | Change percentage (%) |
|---|---|---|---|
| | 8 wks | 16 wks | 16-8 wks |
| ND | 0 | 0 | 0 |
| HFD | 48 | 64 | 16 |
| AS-CH or CH | | | |
| CH (L) | 20 | 27 | 8 |
| CH (H) | 20 | 31 | 11 |
| AS-CH (L) | 25 | 31 | 6 |
| AS-CH (H) | 23 | 30 | 8 |

SD rat was orally administered with various dosages AS-CH (10 or 25 mg/kg BW) for 8 wks. Data is expressed as means ± SD (n = 8).

TABLE 8
Effects of AS-CH on the feed bioavailability in HFD rats

| | Feed bioavailability |
|---|---|
| | % |
| ND | 363.9 |
| HFD | 1029.8 |
| AS-CH or CH | |
| CH (L) | 967.5 |
| CH (H) | 1091.2 |
| AS-CH (L) | 965.2 |
| AS-CH (H) | 1232.4 |

SD rat was orally administered with various dosages AS-CH (10 or 25 mg/kg BW) for 8 wks. Data is expressed as means ± SD (n = 8).

TABLE 9
Effects of AS-CH on the organ weight in HFD rats

| | Heart | Liver | Spleen | Kidney |
|---|---|---|---|---|
| | % of body weight | | | |
| ND | 0.29 ± 0.02 | 2.88 ± 0.09^{b} | 0.14 ± 0.02 | 0.69 ± 0.01 |
| HFD | 0.25 ± 0.03 | 3.45 ± 0.23^{a} | 0.10 ± 0.01 | 0.62 ± 0.04 |
| AS-CH or | | | | |
| CH | | | | |
| CH (L) | 0.29 ± 0.02 | 2.90 ± 0.06^{b} | 0.12 ± 0.02 | 0.62 ± 0.09 |
| CH (H) | 0.27 ± 0.02 | 3.18 ± 0.19^{ab} | 0.14 ± 0.02 | 0.62 ± 0.04 |
| AS-CH (L) | 0.28 ± 0.02 | 2.97 ± 0.09^{b} | 0.13 ± 0.02 | 0.60 ± 0.02 |
| AS-CH (H) | 0.29 ± 0.03 | 2.94 ± 0.20^{b} | 0.12 ± 0.02 | 0.64 ± 0.07 |

SD rat was orally administered with various dosages AS-CH (10 or 25 mg/kg BW) for 8 wks. Data is expressed as means ± SD (n = 8). Significance of difference in activities of different compounds was evaluated by Tukey's test statistical analysis. Different superscript letters^{a,b,c} organs weight are statistically different from each other (p < 0.05).

The above results indicated that the administration of the aqueous soluble-chitosan of the present invention did not cause significant change in food intake, body weight gain, feed bioavailability and organ weight.

### [Analysis for Body Fat Gain and Lipase Activity]

The adipose tissues surrounded kidney and testis were collected and weighted. For determining the activity of lipase, 0.1 gram of the adipose tissue surrounded testis was washed with saline and dried by using filter paper. The washed tissues were homogenized by a homogenizer and then put into centrifugation. After centrifugation, the supernatant was taken for determining the activity of lipase. The results are showed in the following Table 10, Table 11 and FIG. 2 (ND: normal diet; HFD: high fat diet; CH: chitosan (unmodified); AS-CH: aqueous soluble-chitosan (the present invention); L: low dosage (10 mg/kg BW); H: high dosage (25 mg/kg BW)).

TABLE 10
Effects of AS-CH on the body fat in HFD rats

| | Body fat |
|---|---|
| | % of body weight |
| ND | 3.30 ± 1.35^{d} |
| HFD | 12.14 ± 1.66^{a} |
| AS-CH or CH | |
| CH (L) | 8.33 ± 1.60^{bc} |
| CH (H) | 8.80 ± 1.00^{b} |
| AS-CH (L) | 7.66 ± 1.26^{bc} |
| AS-CH (H) | 6.21 ± 1.99^{c} |

SD rat was orally administered with various dosages AS-CH (10 or 25 mg/kg BW) for 8 wks. Data is expressed as means ± SD (n = 8). Significance of difference in activities of different compounds was evaluated by Tukey's test statistical analysis. Different superscript letters^{a,b,c} organs weight are statistically different from each other (p < 0.05).

TABLE 11
Effects of AS-CH on the lipase activity in HFD rats

| | Lipase activity |
|---|---|
| | U/L |
| ND | 5.40 ± 1.46^{b} |
| HFD | 2.18 ± 0.61^{c} |
| AS-CH or CH | |
| CH (L) | 5.05 ± 1.23^{b} |
| CH (H) | 6.46 ± 1.02^{ab} |
| AS-CH (L) | 6.23 ± 0.97^{ab} |
| AS-CH (H) | 8.71 ± 0.54^{a} |

SD rat was orally administered with various dosages AS-CH (10 or 25 mg/kg BW) for 8 wks. Data is expressed as means ± SD (n = 8). Significance of difference in activities of different compounds was evaluated by Tukey's test statistical analysis. Different superscript letters^{a,b,c} intestinal physiology are statistically different from each other (p < 0.05).

It was noted that the aqueous soluble-chitosan of the present invention had a dosage-dependent effect on reducing body fat. This effect may due to its function on increasing the activity of adipose lipase (see Table 11).

Those having ordinary skill in the art can understand various modifications according to the disclosed embodiments without departing from the spirit of the present invention. Therefore, the above-recited embodiments shall not be used to limit the present invention but shall intend to cover all modifications under the spirit and scope of the present invention along with the attached claims.

What is claimed is:

1. A composition for body fat consumption, comprising:
0.1 to 80 wt % of an aqueous soluble-chitosan; and
1 to 50 wt % of a pharmaceutically acceptable carrier.

2. The composition according to claim 1, wherein said aqueous soluble-chitosan has a molecular weight of 0.3 to 1,500 kDa.

3. The composition according to claim 1, wherein said aqueous soluble-chitosan is a chitosan modified by alkyl sultone.

4. The composition according to claim 1, wherein said alkyl sultone is 1,3-propanesultone, 1,4-propylenesultone, 1,4-butanesultone, 2,4-butanesultone, or a mixture thereof

5. The composition according to claim 1, wherein said aqueous soluble-chitosan is a sulfonic acid-modified chitosan.

6. A method for increasing the activity of lipase, comprising: applying a subject in need an effective amount of an aqueous soluble-chitosan.

7. The method according to claim 6, wherein said aqueous soluble-chitosan has a molecular weight of 0.3 to 1,500 kDa.

8. The method according to claim 6, wherein said aqueous soluble-chitosan is a chitosan modified by alkyl sultone.

9. The method according to claim 8, wherein said alkyl sultone is 1,3-propanesultone, 1,4-propylenesultone, 1,4-butanesultone, 2,4-butanesultone, or a mixture thereof

10. The method according to claim 6, wherein said aqueous soluble-chitosan is a sulfonic acid-modified chitosan.

11. The method according to claim 3, wherein said effective amount is 1 to 500 mg/kg BW.

12. The method according to claim 6, wherein said lipase is adipose lipase.
